(19) United States
(12) Patent Application Publication
(10) Pub. No.: US 2013/0125891 A1
(43) Pub. Date: May 23, 2013

(54) SYSTEM AND METHOD FOR DYNAMIC REGULATION OF OXYGEN FLOW RESPONSIVE TO AN OXIMETER

(71) Applicant: Patrick E. Eddy, Allendale, MI (US)

(72) Inventor: Patrick E. Eddy, Allendale, MI (US)

(21) Appl. No.: 13/298,327

(22) Filed: Nov. 17, 2011

(51) Int. Cl. A61M 16/00

(52) U.S. Cl. 128/204.23

(57) ABSTRACT

A system is provided for regulating a flow of oxygen to a patient where the system may include an oximeter for measuring the patient's blood oxygen level and/or heart rate; a controller for determining a desired oxygen flow rate based on the patient's blood oxygen level and/or heart rate, and generating a control signal representative of the desired oxygen flow rate; an oxygen flow regulator for dynamically regulating a flow rate of oxygen supplied to the patient responsive to the control signal provided by the controller; an oxygen delivery device; and a transmitter for wirelessly communicating with an electronic medical records system. The oxygen flow regulator may receive oxygen from an oxygen bottle, oxygen mixer, a wall oxygen supply of a healthcare facility, or an oxygen concentrator. One or more of the components of the system may be treated with an antimicrobial material.

### BACKGROUND OF THE INVENTION

The embodiments below generally pertain to a system and method for regulating a flow of oxygen to a patient.

Certain diseases and deficiencies of the respiratory system can cause blood oxygenation to drop, causing breathing difficulty, fatigue or more serious problems for the patient. Application of pure oxygen is used to treat these conditions; an oxygen flow from a bottled source or other source is given to the patient through a mask or nose piece. However, not all of the oxygen is required at all times. Use of oxygen changes with patient activity similarly to increases or decreases in pulse rate.

Normally, a static oxygen flow rate is prescribed to the patient, giving them excess oxygen when they are inactive. This wastes oxygen thereby increasing the cost to the patient and the time required to obtain and switch oxygen bottles.

### SUMMARY OF THE INVENTION

According to an embodiment of the present invention, a system for regulating a flow of oxygen to a patient is provided comprising: an oximeter for measuring at least one of a patient's blood oxygen level and heart rate; a controller for acquiring the patient's blood oxygen level/heart rate from the oximeter and determining a desired oxygen flow rate based on the measured blood oxygen level/heart rate; and an oxygen flow regulator for dynamically regulating a flow rate of oxygen supplied to the patient responsive to a control signal provided by the controller.

According to another embodiment of the present invention, an integrated oximeter and oxygen delivery device comprise: a nasal cannula having an oxygen delivery hose for delivering oxygen to a patent's nose; and an oximeter integrated with the oxygen delivery hose for securing to the patent's septum to read the patient's blood oxygen level.

According to another embodiment of the present invention, a method for regulating a flow of oxygen to a patient is provided comprising: continuously measuring at least one of a patient's blood oxygen level and heart rate; automatically determining a desired oxygen flow rate based on the measured blood oxygen level/heart rate; and dynamically regulating a flow rate of oxygen supplied to the patient responsive to the determined desired oxygen flow rate.

These and other features, advantages, and objects of the present invention will be further understood and appreciated by those skilled in the art by reference to the following specification, claims, and appended drawings.

### BRIEF DESCRIPTION OF THE DRAWINGS

In the drawings:

FIG. 1 is a block diagram generally showing an embodiment of the present invention;

FIG. 2 is a block diagram showing components of a controller that may be used in the embodiment shown in FIG. 1;

FIG. 3 is a perspective view of an integrated oximeter and oxygen delivery device that may be used in the embodiment shown in FIG. 1;

FIG. 4 is a perspective view of the integrated oximeter and oxygen delivery device of FIG. 3 shown in use on a patient;

FIG. 5 is a perspective view of an oxygen flow regulator that may be used in the embodiment shown in FIG. 1;

FIG. 6 is an isometric view of a plate used in the oxygen flow regulator shown in FIG. 5;

FIG. 7 is an electrical schematic diagram of circuitry forming a power supply circuit that may be used in the embodiment shown in FIGS. 1 and 2 and that was used in a prototype described as the Example below;

FIG. 8 is an electrical schematic diagram of a microcontroller that may be used in the embodiment shown in FIGS. 1 and 2 and that was used in a prototype described as the Example below;

FIG. 9 is an electrical schematic diagram of circuitry forming an oximeter sensor interface that may be used in the embodiment shown in FIGS. 1 and 2 and that was used in a prototype described as the Example below; and

FIG. 10 is an electrical schematic diagram of circuitry forming an oxygen flow regulator interface that may be used in the embodiment shown in FIGS. 1 and 2 and that was used in a prototype described as the Example below.

### DETAILED DESCRIPTION OF PREFERRED EMBODIMENTS

Reference will now be made in detail to the present preferred embodiments of the invention, examples of which are illustrated in the accompanying drawings. Wherever possible, the same reference numerals will be used throughout the drawings to refer to the same or like parts. In the drawings, the depicted structural elements are not to scale and certain components are enlarged relative to the other components for purposes of emphasis and understanding.

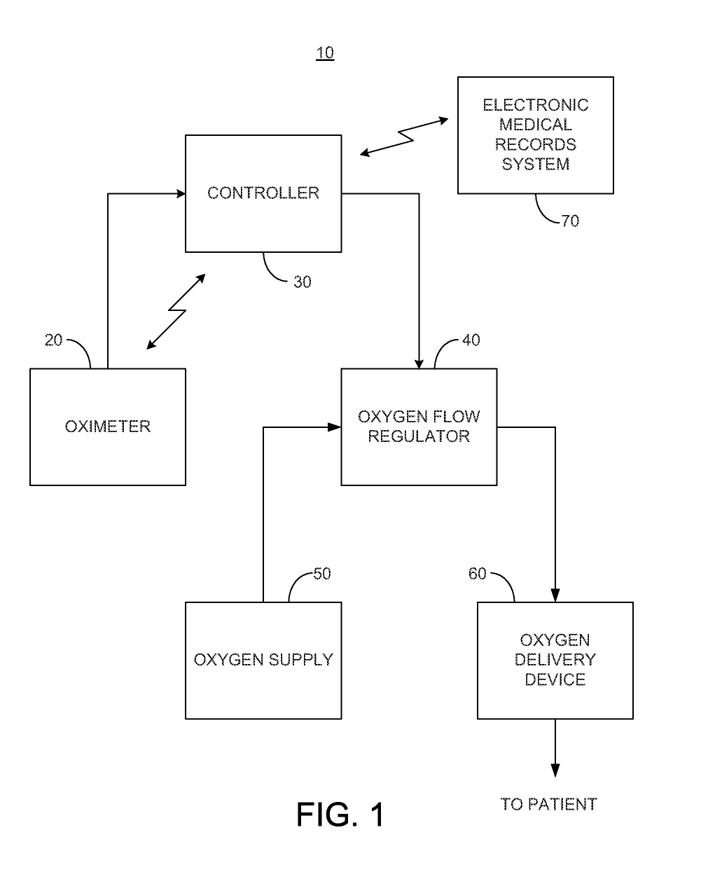

As shown in FIG. 1, a system 10 is provided that generally includes an oximeter 20, a controller 30, and an oxygen flow regulator 40 that regulates the flow of oxygen from an oxygen supply 50 to a patient 80. As described in detail below, either or both of the patient's blood oxygen level and heart rate is measured by oximeter 20. Controller 30 acquires the patient's blood oxygen level/heart rate from oximeter 20 and controls oxygen flow regulator 40 to dynamically regulate the supply of oxygen to the patient responsive to the patient's blood oxygen level and/or heart rate.

Oximeter 20 may be a pulse oximeter or any other form of oximeter capable of reading a patient's blood oxygen level and optionally the patient's heart rate, and outputting a representative signal to a controller. Pulse oximeters are known and typically employ differing visible/infrared light transmission of oxygenated/deoxygenated to measure the percentage of blood that is carrying oxygen. Known oximeters take readings from a patient's fingers, toes, or ears. As mentioned below, they can also be configured to take readings from the patient's septum. To the extent the oxygen flow is regulated based on heart rate alone, a heart rate monitor may be used in place of the oximeter.

Controller 30 reads data from oximeter 20 and converts the data into the preferred oxygen flow rate. Such conversion may be by use of a lookup table. Using a flow-rate lookup table allows maximum flexibility over the oxygen prescribed to the patient. Controller 30 may be coupled to oximeter 20 by a wire or by wireless communication. Wireless communication between oximeter 20 and controller 30 may take any form of wireless technology and communication protocol, such as a BLUETOOTH™ protocol.

In addition, controller 30 may optionally be configured to wirelessly communicate with external systems such as those in a hospital or other care facility. One example of such an external system is an Electronic Medical Records (EMR) system 70 such as a Cerner records database. With such a wireless communication to an EMR system, if a patient used the device, the wireless feature could help record the oxygen levels and heart rates as read by the oximeter as well as the oxygen flow rate determined by the controller.

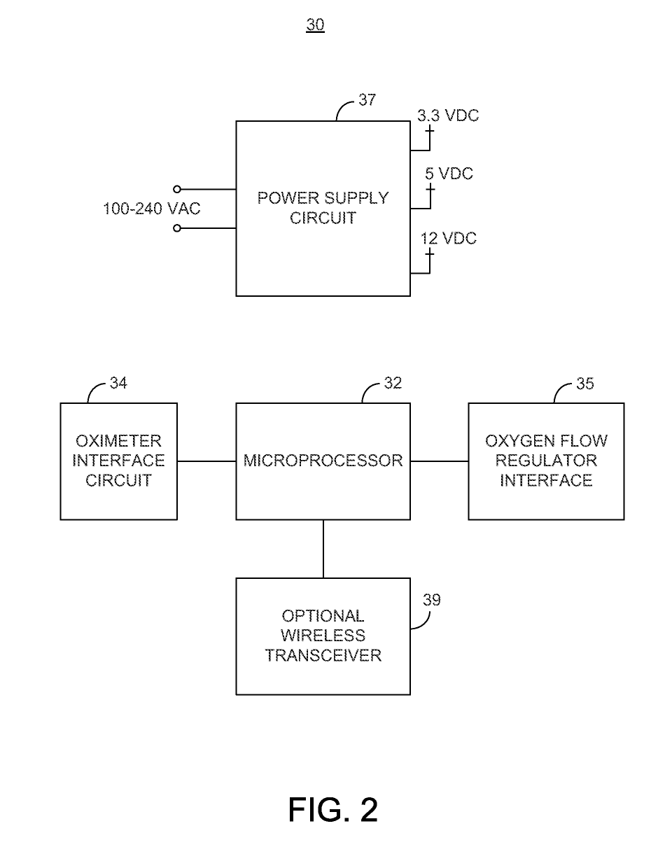

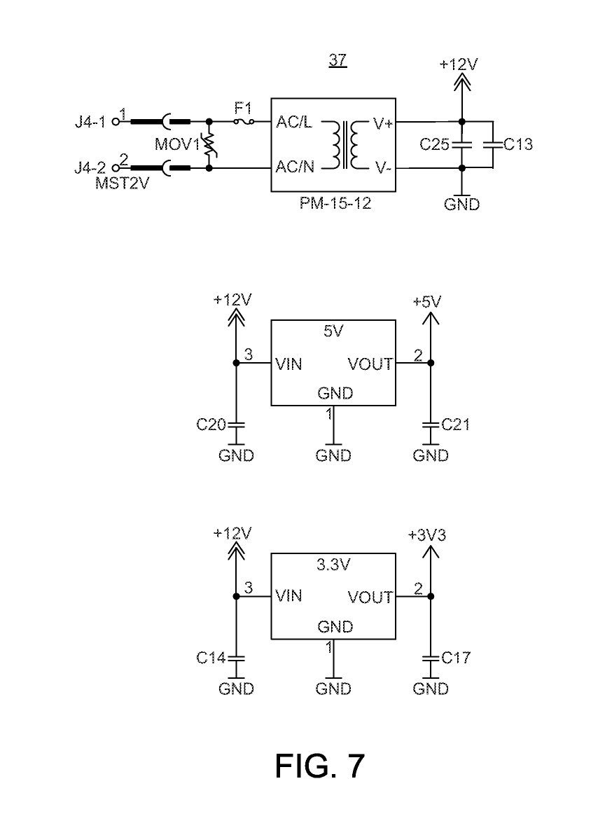

FIG. 2 shows a block diagram of an exemplary controller 30. As shown, the controller 30 may include a microprocessor or microcontroller 32, an oximeter interface circuit 34, an oxygen flow regulator interface 35, a power supply circuit 37, and an optional wireless transceiver 39. Examples of the construction of power supply circuit 37, microcontroller 32, oximeter interface circuit 34, and oxygen flow regulator interface 35 are provided below in association with FIGS. 7-10, respectively.

Oxygen flow regulator 40 may include any electrically controlled valve having sufficient precision to vary the oxygen flow rate over the variable increments desired. For example, oxygen flow regulator may include an electronically controlled valve such as an “O-E” Series or Oxygen Series pneumatic valves available from Clippard Instrument Laboratory, Inc. of Cincinnati, Ohio. In the example provided below, oxygen flow regulator 40 may be implemented using a stepper motor connected to a commercially available manual medical oxygen flow regulator. Changing the angle of the shaft of the stepper motor changes the output flow rate from the regulator.

Oxygen supply 50 may be any form of oxygen supply such as an oxygen bottle, oxygen concentrator, oxygen mixer, or a wall supply in a healthcare facility.

The advantage of a dynamically adjusting oxygen flow is that oxygen is conserved when it is not required by the patient, but is supplied in prescribed quantities when needed. This extends the life of an oxygen bottle and can greatly reduce the amount of oxygen consumed, saving both time to exchange bottles and money in replenishing the oxygen supply.

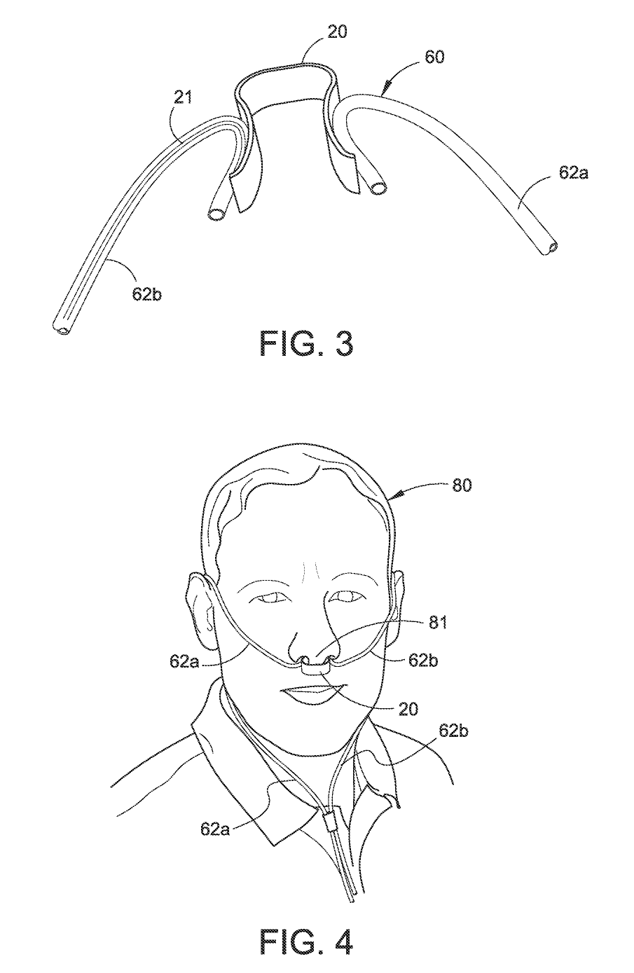

The oxygen can be delivered by oxygen delivery device 60 (FIGS. 1, 3, and 4), which may be a nasal cannula or a mask. As noted above, oximeter 20 may be integrated with oxygen delivery device 60. For example, as shown in FIG. 3, oxygen delivery device 60 includes a nasal cannula formed at the end of one or more oxygen delivery hoses 62a and 62b. As also shown in FIG. 3, an oximeter 20 is provided in the form for measuring the blood oxygen level at a patient's septum and is integrated with oxygen supply hoses 62a and 62b with oximeter wires 21 extending along one of hoses 62a and 62b. FIG. 4 shows the integrated oximeter and oxygen delivery device 60 secured to a septum 81 of a patient 80. Thus, if oxygen is to be delivered into the patient's nose, measurements could be taken from the septum of the nose, requiring only minor changes in the form factor of existing oxygen delivery nose pieces. Thus, the same disposable device could be used to measure the blood oxygen level at the septum and to deliver the oxygen.

One or more of the nasal cannula, mask, oximeter, and oxygen flow regulator whether used with bottles, concentrators, or wall supplies, may be treated with an antimicrobial material. Such an antimicrobial material may be Microguard® (by Microguard, Olivet, France), which is a liquid solution containing hydrophilic polymers, or Microban® antimicrobial plastic additive available from Microban International. A preferred antimicrobial material is MicrobeCare™ (from MicrobeCare, LLC of Allendale, Mich.), which is a copolymer of chloropropyltrihydroxysilane and octadecylaminodimethyltrihydroxysilylpropyl ammonium chloride. Other antimicrobial substances include 3 trimethoxysilylpropyloctadecyldimethyl ammonium chloride, hyaluronan and its derivatives, triclosan, and an organosilicon antimicrobial that is substantially free from arsenic, silver, tin, heavy metals and polychlorinated phenols. The antimicrobial substance could be copper or a silver-ion emitter. One silver-ion emitter is Germ-Gate™ (from Bovie Screen Process Co., Inc., Bow, N.H.), which is a nano particle silver based, liquid coating that can be coated onto a fabric. Another silver-ion emitter is ProtexAG (from Carolina Silver Technologies, North Carolina), which is a silver-based coating that can be coated onto fabric. Yet other silver-ion emitting coatings are those available from Covalon Technologies, Ltd. of Mississauga, Ontario, Canada; Agion® antimicrobial coating available from Agion Technologies Ltd. of Wakefield, Mass.; and Zeolite carrying silver, Model No. XDK101 available from Xiamen Xindakang Inorganic Materials Co., Ltd. In addition, silver sodium hydrogen zirconium phosphate may be used as the antimicrobial substance 36. Alternate antimicrobial materials may be used that are tolerant of appropriate cleaning and sterility methods, an example of which is zirconium phosphate such as Model No. XDK801 available from Xiamen Xindakang Inorganic Materials Co., Ltd. In general terms, an antimicrobial substance is capable of emitting ions that aid in the destruction of a microbe.

### EXAMPLE

The following description is provided to illustrate one exemplary prototype of the system described above. This example is not intended to be limiting.

In this example, oximeter 20 was implemented using a pulse oximeter sensor and more particularly, the commercially available CMS-50 fingertip pulse oximeter available from Contec Medical Systems Co., Ltd. of Qinhuangdao, China. This sensor clips to the fingertip of the patient and calculates patient pulse rate and oxygen concentration. This data is transmitted to the controller circuitry via a UART interface where it is used to calculate flow rate data. The CMS-50 sensor was chosen for its wide commercial availability and its plug and play nature. The CMS-50 only needs to be connected to the controller with the proper USB cable. In this example, a standard USB A to mini B cable was used.

The controller 30 circuitry provides the means to calculate the oxygen flow rates, integrate the sensor readings, and actuate the flow regulator. In this example, the controller circuitry was a custom design that utilizes a microcontroller 32, oximeter interface circuit 34, oxygen flow regulator interface 35, and a power supply circuit 37. Schematics for the controller circuitry are shown in FIGS. 7-10. In FIGS. 7-9, the following circuit components had the listed parameters:

| | | |
|---|---|---|
| C1 | 0.1 | μF |
| C2 | 0.1 | μF |
| C9 | 0.1 | μF |
| C10 | 0.1 | μF |
| C11 | 0.1 | μF |
| C13 | 0.1 | μF |
| C14 | 0.1 | μF |
| C17 | 0.1 | μF |
| C20 | 0.1 | μF |
| C21 | 0.1 | μF |
| C23 | 10 | μF |
| C25 | 470 | μF |
| D1 | 6 | V |
| R23 | 10 | kΩ |

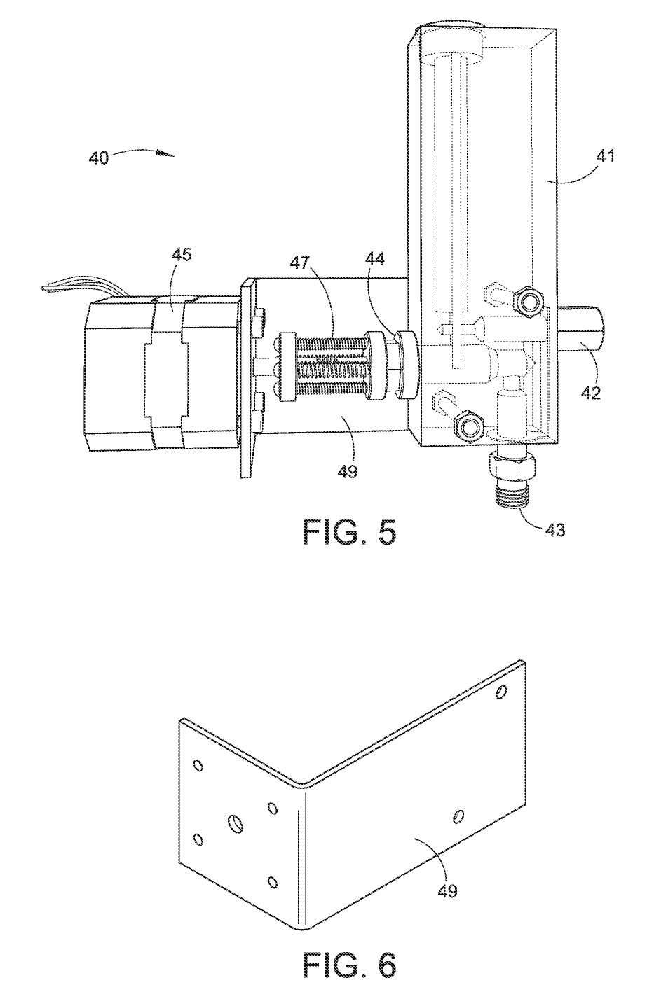

In this example, the oxygen flow regulator 40 was implemented with a standard, off-the-shelf medical gas flow regulator 41 as shown in FIG. 5. The gas flow regulator 41 was attached directly to the oxygen supply at 50 PSI via inlet 42 and is manually adjusted to change the output flow of oxygen by rotating an adjustment knob shaft 44.

To adjust the flow under electronic control, a stepper motor 45 was attached to the adjustment knob shaft 44. A customized spring-loaded shaft coupling 47 accommodates the thrust motion of the adjustment knob shaft 44, connecting the motor 45 and adjustment shaft 44. The stepper motor 45 is commanded by the controller circuitry 30 to a certain angle, finely controlling the output oxygen flow from outlet 43.

The prototype was designed to run continuously and therefore runs on line voltage (100-240VAC). To eliminate complexity and possible failure points from the design, a pre-designed isolated, medical power supply was selected to generate a 12VDC power rail to power the logic and motor drive circuitry.

FIG. 7 shows power supply circuit 37. The main power supply circuit chosen is the Mean-Well PM-15-12 available from MeanWell Enterprises Co., Ltd. of Taiwan. This power supply comes with all of the needed certifications for integration into a medical product.

As shown in FIG. 7, logic power supplies of 5V and 3.3V are generated onboard from the 12V main power rail. Linear regulators are used for simplicity and for cost concerns. A switch-mode power supply could be used in this situation.

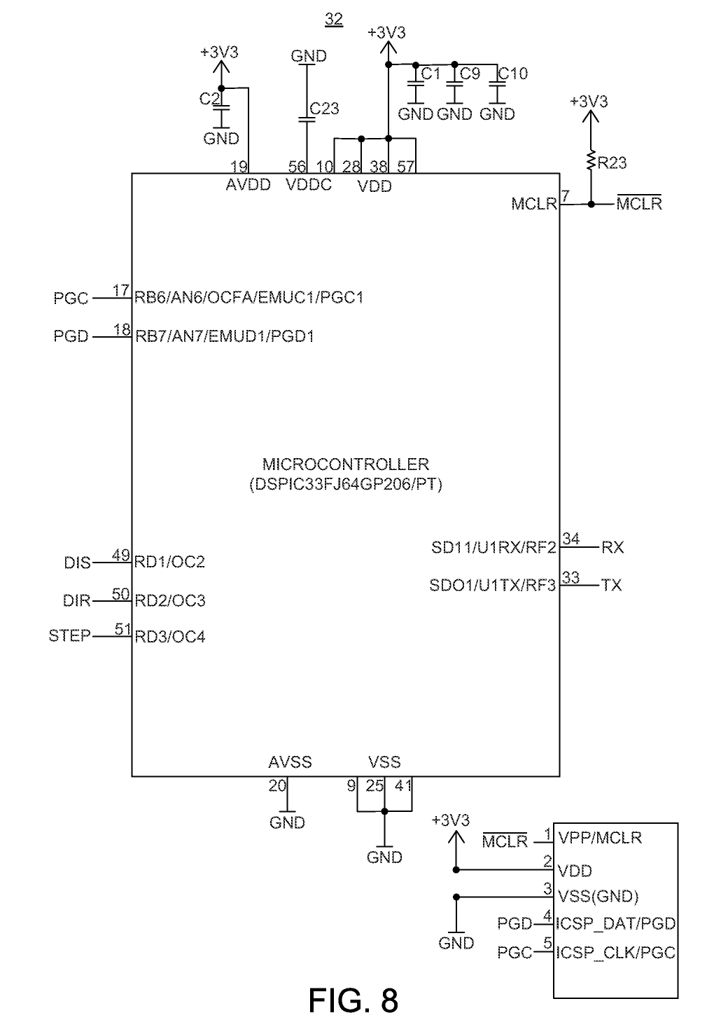

Microcontroller 32 is shown in FIG. 8. The microcontroller selected to control the operation of the prototype was the dsPIC33FJ64GP206 microcontroller available from Microchip Technology Inc. of Chandler, Ariz. This microcontroller was selected for several reasons including: CMOS 3.3V voltage level, suitable for interface with the CMS-50 UART port; a large program memory and RAM space available for debugging; and two available UARTs for communication with the pulse oximeter sensor and a debug output.

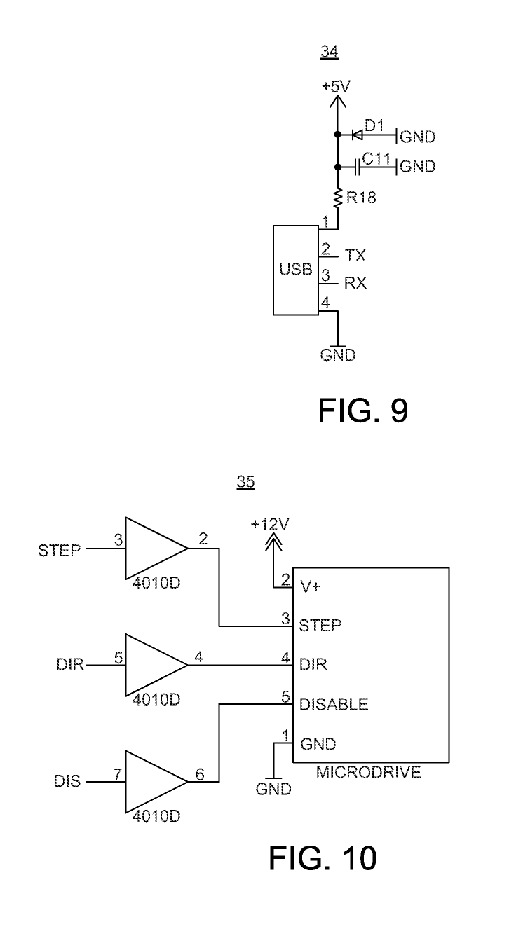

The microcontroller is connected to two circuits and is responsible for their operation. The first circuit is the UART communication with the oximeter also referred to herein as oximeter interface circuit 34, which is shown in FIG. 9. Some interface circuitry is present at the connector to provide power to the oximeter and prevent incorrect devices from being damaged or damaging the prototype. The second circuit is a GPIO buffer that connects the controller to an external motor controller circuit. The GPIO buffer is also referred to as the oxygen flow regulator interface 35 and is shown in FIG. 10.

The CMS-50 pulse oximeter normally connects to a PC host which recharges it and collects pulse and oxygen concentration data; graphing it was requested by the user.

The CMS-50 connects through what appears to be a USB interface. However, the CMS-50 actually exposes an RS-232 TX/RX pair on the USB mini-B connector as shown in FIG. 9. Further investigation shows that the cable included with the CMS-50 is actually an RS-232 to USB converter (see above). By using a normal USB cable, the RS-232 lines are directly connected to the controller without being converted to USB.

A 5V and ground line are in the standard pin locations for the USB-mini B connector. Applying a voltage to these lines recharges the CMS-50.

The UART physical protocol used to transmit the pulse/ox data is 8N1 at 19200 baud. The data is transmitted in a repeating datagram, 2 bytes followed by 3 bytes several milliseconds later. The first byte of the 2 byte section is the pulse rate from 0-255, the second byte is the blood oxygen level, 0-100. The other 3 bytes probably represent control data used by the PC program the user is meant to use; however, they do not contain any relevant data for this application. If the sensor is removed or does not detect any pulse/ox data, it stops transmitting.

By continuously receiving these datagrams, the prototype can constantly calculate the appropriate motor shaft angle to produce an oxygen flow.

The stepper motor drive in FIG. 10 provides the electrical interface 35 and driving capability to run the actuator stepper motor 45 (FIG. 5). The stepper drive is a Microdrive available from Selene Photonics Inc. of Houghton, Mich. It is a small form factor driver meant for small actuator motors. The driver presents a simple interface 35 to the prototype microcontroller 32 allowing it to be used with GPIO alone.

The driver is powered by the main bus voltage. Control/logic signals are TTL voltage level and are generated on the driver board itself (no TTL power bus connection is required).

As shown in FIG. 10, the stepper controller interface 35 of the prototype has three inputs STEP, DIR and DIS that are received from microcontroller 32 (FIG. 8).

The STEP input command is positive edge sensitive. An incoming edge will signal the driver to move the motor shaft a full size step (1.8 degrees). Certain setup and hold times are required for an edge to be registered. The microcontroller meets these constraints by holding the output for several milliseconds before releasing it.

The DIR signal is level sensitive. The logical level of the DIR signal when a STEP input command is detected determines the direction that the motor steps. The actual direction the motor 45 rotates depends on the wiring configuration/connection to the driver.

Lastly, the DIS signal, also level sensitive, disables or enables the motor drive outputs. A level of 1 on the DIS line sets the motor into operation. The driver will attempt to actively hold the motor position. This is, of course, accompanied by power dissipation in the motor with some audible noise. A 0 level on this line disables the motor outputs. No step commands are accepted in this condition.

The firmware for the controller is implemented in C for use on the MPLAB/C30 platform by Microchip Technology. The code targets the dsPIC33FJ64GP206 microcontroller.

The firmware operated in two stages, initialization and operation. The initialization stage occurs first, just after the device is powered up. Apart from basic initialization procedures to power up the microcontroller and the needed peripherals, the controller turns the motor several rotations in an attempt to set the shaft into a known position. The controller will turn the actuator shaft farther than its maximum possible range, guaranteeing that the actuator is in the full closed position. This method of homing the actuator and using open loop control for subsequent operations works properly, but could be replaced by an angular feedback system.

General operation was done in a continuous loop. Measurements were taken from the CMS-50 pulse oximeter, actuator commands were calculated and then executed. Any change in sensor readings precipitated an actuator response.

Output oxygen flow was controlled by a lookup table in the firmware of the controller. Tuning these levels in the prototype device could be done by changing the values in firmware. The code:

#define MAX_OX 100
#define MIN_OX 90
INT16 lookup[MAX_OX − MIN_OX] = {...};

contains the lookup table. The lookup table contains angles to set the actuator shaft from its home position (0).

The definition MAX_OX was the blood oxygen level where the patient will receive the least additional oxygen. Similarly, the MIN_OX definition was the blood oxygen concentration where the patient will receive maximum additional oxygen. The values in the lookup table all corresponded to the integer concentrations between the minimum and maximum. If the sensor read a value beyond the minimum or maximum concentration values, the lowest or highest index in the lookup table, respectively, would be used.

To eliminate issues where the angle of the motor shaft (and thus the output flow) becomes disrupted, then throwing off all subsequent changes in flow output, a shaft position feedback sensor could be used to report the actual shaft position. This could be done with a simple potentiometer integrated into the regulator.

The prototype used a preprogrammed prescription. A certain oxygen flow was given for a certain blood oxygen concentration. No user interface was given. All internal variables are controlled by this prescription. A user interface could be provided that the patient or doctor could change the prescription, changing the maximum and minimum oxygen flows with the corresponding blood oxygen levels to dispense those flows.

The prototype mechanical actuator was created from several off-the-shelf components. A similar actuator could have similar components integrated into a single device.

The prototype actuator was comprised of a manual medical oxygen flow meter and stepper motor. The oxygen flow meter was designed to be adjusted manually using an adjustment knob that changes the oxygen flow rate. The stepper motor in the prototype was attached through a specialized coupling to turn the adjustment shaft electromechanically to allow electronic control over the oxygen flow.

A folded steel frame 49 as shown in FIGS. 5 and 6 connects the stepper motor 45 and the gas flow regulator 41.

The stepper motor 45 used was also an off-the-shelf NEMA17 sized motor. Its model number is ROB-09238 and it had the following specifications:

Step Angle: 1.8 degrees (200 steps/rev)

Rated Current: 0.33 A

Shaft Diameter: 5 mm

Holding Torque: 2.3 Kg*cm

The motor was selected for its relatively small form factor, low current requirements and adequate torque. Electrically, the motor was a two phase, 4-wire bipolar motor. The wiring configuration allowed the motor to connect directly to the Selene Microdriver motor outputs.

As mentioned above, coupling 47 was used between the stepper motor 45 and the flow regulator shaft 44 that could absorb the thrust motion of the regulator shaft without stressing any of the components. The coupling is shown in FIG. 5. The coupling consisted of two, 5 mm shaft couplings that attached by set screw to the motor's host shaft and by 4 4-40 tapped machine screws to their load. One of the 4-40 screw threads of the coupling was removed so a machine screw could slide freely in the axial direction through it. The two couplings were attached with a series of 4-40×1.25″ screws. Two springs are used to provide a thrust preload. Radial torque was transmitted, but thrust movement was absorbed by the coupling.

The control circuitry was designed to fit into an RS-4025 extruded aluminum enclosure manufactured by X-Tech (xtech-outside.com). The circuit board slid into pre-extruded slots in the enclosure.

The prototype described above was intended to illustrate only one example of how the system could be implemented.

The oximeter used in the example above was a standalone, off-the-shelf component attached via an external interface to the controller. However, the oximeter could be integrated with the controller. This would reduce production and procurement costs as well as reduce the number of components of the device.

The system may be configured to include fail-safe procedures to prevent accidental injury. For example, on signal loss from the sensor, the actuator could output maximum prescribed oxygen. This will ensure that on failure of the sensor, oxygen will still be supplied to the patient. Further, a spring mechanism could be employed to ensure that the valve is forced open when an external force is not applied. This ensures that if the electronic actuator fails, an oxygen supply is still available. This also requires that the electronic actuator always works to close the oxygen supply, fulfilling its purpose to conserve oxygen (rather than dispensing it, as a normally closed version would).

The above description is considered that of the preferred embodiment(s) only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

The invention claimed is:

1. A system for regulating a flow of oxygen to a patient comprising:
an oximeter for measuring at least one of the patient's blood oxygen level and heart rate;
a controller for acquiring the patient's blood oxygen level/heart rate from said oximeter, determining a desired oxygen flow rate based on the patient's blood oxygen level/heart rate, and generating a control signal representative of the desired oxygen flow rate; and
an oxygen flow regulator for dynamically regulating a flow rate of oxygen supplied to the patient responsive to the control signal provided by said controller.

2. The oxygen flow regulating system of claim 1, wherein said oxygen flow regulator is configured for regulating the flow rate of oxygen received from an oxygen bottle.

3. The oxygen flow regulating system of claim 1, wherein said oxygen flow regulator is configured for regulating the flow rate of oxygen received from a wall oxygen supply of a healthcare facility.

4. The oxygen flow regulating system of claim 1, wherein said oxygen flow regulator is configured for regulating the flow rate of oxygen received from an oxygen concentrator.

5. The oxygen flow regulating system of claim 1, wherein said oximeter is a pulse oximeter.

6. The oxygen flow regulating system of claim 1, wherein said oximeter communicates wirelessly with said controller.

7. The oxygen flow regulating system of claim 1, wherein said controller includes a transmitter for wirelessly communicating with an electronic medical records system, wherein said transmitter transmits at least one of the patient's blood oxygen level and the oxygen flow rate to the electronic medical records system.

8. The oxygen flow regulating system of claim 1 and further comprising an oxygen delivery device comprising one of a nasal cannula and a mask.

9. The oxygen flow regulating system of claim 8, wherein at least one of said oximeter, said oxygen flow regulator, and said oxygen delivery device is treated with an antimicrobial material.

10. The oxygen flow regulating system of claim 8, wherein said oxygen delivery device comprises a nasal cannula having an oxygen delivery hose, and wherein said oximeter is configured to secure to the patient's septum and is integrated with said oxygen delivery hose.

11. An integrated oximeter and oxygen delivery device comprising:
a nasal cannula having an oxygen delivery hose for delivering oxygen to a patient's nose; and
an oximeter integrated with said oxygen delivery hose for securing to the patient's septum to read the patient's blood oxygen level.

12. The integrated oximeter and oxygen delivery device of claim 11 further comprising a wire for connecting to said oximeter, said wire being secured to said oxygen delivery hose.

13. The integrated oximeter and oxygen delivery device of claim 11 further comprising a second oxygen delivery hose integrated with said oximeter.

14. A method for regulating a flow of oxygen to a patient comprising:
continuously measuring at least one of a patient's blood oxygen level and heart rate;
automatically determining a desired oxygen flow rate based on the measured blood oxygen level/heart rate; and
dynamically regulating a flow rate of oxygen supplied to the patient responsive to the determined desired oxygen flow rate.

15. The method of claim 14 and further comprising providing an oxygen bottle, wherein the step of dynamically regulating a flow rate of oxygen regulates the flow rate of oxygen received from the oxygen bottle.

16. The method of claim 14, wherein the step of dynamically regulating a flow rate of oxygen regulates the flow rate of oxygen received from a wall oxygen supply of a healthcare facility.

17. The method of claim 14, wherein the step of dynamically regulating a flow rate of oxygen regulates the flow rate of oxygen received from an oxygen concentrator.

18. The method of claim 14, wherein the oximeter is a pulse oximeter.

19. The method of claim 14, wherein the oximeter communicates wirelessly.

20. The method of claim 14 and further comprising wirelessly transmitting at least one of the patient's blood oxygen level and the oxygen flow rate to an electronic medical records system.
